(19) United States
(12) Patent Application Publication
(10) Pub. No.: US 2010/0234468 A1
(43) Pub. Date: Sep. 16, 2010

(54) NOVEL PROCESS

(76) Inventors: Abhay Gaitonde, Maharashtra (IN); Bindu Manojkumar, Maharashtra (IN); Sandeep Mekde, Maharashtra (IN); Dattatrey Kokane, Maharashtra (IN)

Correspondence Address: FULBRIGHT & JAWORSKI L.L.P.;Attn: MN IP Docket, 600 Congress Avenue, Suite 2400, Austin, TX, 78701, US

(21) Appl. No.: 12/602,939

(22) PCT Filed: May 30, 2008
(86) PCT No.: PCT/GB08/50397

§ 371 Date: Jun. 2, 2010

(30) Foreign Application Priority Data
Jun. 4, 2007 (IN) 1038/MUM/2007

(51) Int. Cl. A61K 31/165; C07C 233/11; A61P 25/00

(52) U.S. Cl. 514/618; 564/162

(57) ABSTRACT

The present invention relates to a process for the preparation of polymorphic forms of the R- and S-enantiomers of modafinil, R-(−)-2-benzhydrylsulfinylacetamide and S-(+)-2-benzhydrylsulfinylacetamide respectively.

### CROSS-REFERENCE TO RELATED APPLICATION(s)

This application is a Section 371 National Stage Application of International No. PCT/GB2008/050397, filed 30 May 2008 and published as WO 2008/1419141 A2 on 11 Dec. 2008, which claims priority from the IN Patent Application No. 1038/MUM/2007, filed 4 Jun. 2007, the contents of which are incorporated herein in their entirety for all purposes.

### FIELD OF THE INVENTION

The present invention relates to a process for the preparation of polymorphic forms of the R- and S-enantiomers of modafinil (formula I), R-(−)-2-benzhydrylsulfinylacetamide and S-(+)-2-benzhydrylsulfinylacetamide respectively.

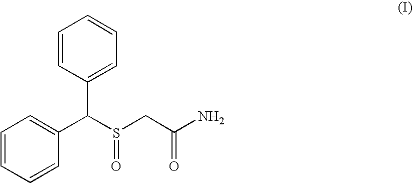

### BACKGROUND OF THE INVENTION

Modafinil is a memory-improving and mood-brightening psychostimulant. It is referred to as a wakefulness-promoting agent and is indicated for the symptomatic relief of excessive sleepiness associated with narcolepsy, obstructive sleep apnoea/hypopnoea syndrome (OSAHS), and moderate to severe chronic shift work sleep disorder (SWSD).

Modafinil is a racemic compound which is chiral at the sulfur atom. The molecule exists as two isomers, R-(−)-modafinil and S-(+)-modafinil. The dextro- and levorotatory enantiomers of modafinil do not interconvert and have different pharmacokinetics. It is further well known in the art that the dextro- and levorotatory enantiomers of modafinil exhibit polymorphism.

Modafinil was first described in U.S. Pat. No. 4,177,290, which relates to 2-benzhydrylsulfinyl-acetamide and processes for its small scale and industrial synthesis. According to U.S. Pat. No. 4,177,290, benzhydrylthioacetic acid was chlorinated with thionyl chloride. The resulting benzhydrylthioacetyl chloride was converted to benzhydrylthioacetamide by reaction with ammonia. The amide was oxidized with acetic acid/hydrogen peroxide to form 2-benzhydrylsulfinylacetamide. The industrial process involved the reaction of thiourea, 48% hydrobromic acid, benzhydrol, and chloroacetic acid to form benzhydrylthioacetic acid. This was oxidized with hydrogen peroxide to give benzhydrylsulfinylacetic acid. This was converted to the methyl ester in the presence of dimethyl sulfate, which on treatment with ammonia in the presence of anhydrous methanol yielded the required product.

U.S. Pat. No. 4,927,855 describes (−)-2-benzhydrylsulfinylacetamide, i.e. the levorotatory enantiomer, and a process for its preparation. The patent also describes the preparation of the dextrorotatory isomer. Racemic benzhydrylsulfinylacetic acid was resolved with (−)-α-methylbenzylamine to yield the levorotatory addition compound. This was hydrolyzed with hydrochloric acid to give the levorotatory isomer of benzhydrylsulfinylacetic acid. This was converted to the methyl ester with methyl sulfate, which on treatment with ammonia gave the final product. The dextrorotatory enantiomer was prepared by resolution with (+)-α-methylbenzylamine. The patent does not disclose or provide any information on purifying the resultant compounds or even allude to the likelihood of the presence of impurities in the final compounds or in the chiral intermediates or the effect of the impure intermediates on the purity of the final compounds.

WO 2004/101503 describes a process for the preparation of modafinil with a definite granulometry. This application describes form I (marketed form) and form III of racemic modafinil, which are interconvertible. The application describes a process for the preparation of racemic modafinil, which comprises the steps of:

(a) preparing a solution of methyl benzhydrylsulfinylacetic acid in a protic polar solvent;
(b) contacting the solution obtained with ammonia under a predetermined temperature and stirring; and
(c) isolating the modafinil formed.

The polar protic solvents employed are methanol, ethanol, propanol, butanol, isobutyl alcohol, t-butyl alcohol, methoxyethanol, ethoxyethanol, pentanol, neopentyl alcohol, t-pentyl alcohol, cyclohexanol, ethylene glycol, propylene glycol, benzyl alcohol, phenol and glycerol, methanol being preferred. The application further discloses a process for the preparation of form I and form III of both the dextro- and levorotatory enantiomer. The invention is directed to modafinil obtainable by the process disclosed above and which has been shown to display a characteristic and reproducible particle size distribution and impurity profile.

WO 2005/077894 describes pharmaceutical compositions comprising modafinil, and methods for preparing the same. The application discloses a composition comprising R-(−)-modafinil and S-(+)-modafinil, a composition comprising R-(−)-modafinil form III, a composition comprising R-(−)-modafinil form IV, and a composition comprising R-(−)-modafinil form V. It further discloses solvents for the crystallization of R-(−)-modafinil. The solvents are selected from acetonitrile, dimethylformamide (DMF), methanol, methyl ethyl ketone, N-methylpyrrolidone, ethanol, isopropanol, isobutanol, formamide, isobutyl acetate, 1,4-dioxane, tetrahydrofuran (THF), ethyl acetate, o-xylene, isopropyl acetate, dichloromethane, propylene glycol, acetic acid, water, acetone, nitromethane, toluene, benzyl alcohol and their mixtures. WO 2005/077894 also describes a process for preparing modafinil form V that involves heating modafinil form IV in ethanol to reflux and then cooling to room temperature. This process specifically requires form IV as the starting material and modafinil does not dissolve during the process; hence complete purification is not possible. Therefore the chemical and optical purity of the product obtained is low. Another process reported in WO 2005/077894 gives form V by heating modafinil in ethyl acetate to 60° C. to get a clear solution, evaporating one third to one half of the solvent using nitrogen flow, cooling to room temperature and then filtering. This process is less feasible practically, since it is not possible to maintain a tight control on the quantity of solvent distilled (especially on plant scale) and the volume of solvent is critical to obtain form V.

U.S. Patent Publication No. 2006/0135621 and WO 2004/060858 describe processes for preparing forms I, II, III, IV, and V of the dextro- and levorotatory enantiomer of modafinil and also solvates of modafinil. The application relates to a process for the preparation of crystalline forms of the optical enantiomers of modafinil characterised by their XRD spectra. Typical solvents for the levorotatory form I include acetone, methanol, ethanol, 1,4-dioxan, ethyl acetate, and mixtures of ortho-, meta- and para-xylene. Typical solvents for the levorotatory form II include isopropanol, ethyl acetate, n-propanol, or ethanol denatured with toluene. Typical solvents for the levorotatory form III include acetone. Typical solvents for the levorotatory form IV include tetrahydrofuran (THF), chloroform, and methyl ethyl ketone. Typical solvents for the levorotatory form V include 2-pentanone and tetrahydrofuran (THF). The present inventors have found that the processes described in US 2006/0135621 and WO 2004/060858 do not give modafinil form II of good optical and chemical purity.

U.S. Patent Publication No. 2005/0038124 describes a process for the preparation of form II by stirring form III in water at pH 5.9 and by filtering. This process specifically requires form III as starting material and modafinil does not dissolve during the process; hence complete purification is not possible. Therefore the chemical and optical purity of the product obtained is low.

Thus there is a long felt need in the art for a process which overcomes all these difficulties.

### SUMMARY OF THE INVENTION

In comparison to the processes disclosed in the prior art, the process of the present invention is operationally simple and does not require a particular modafinil form as starting material. Further, the methods of the present invention of making modafinil forms 2 and 5 involve actual crystallization, which improves the chemical and optical purity. The volume of solvent used in the present invention is almost one half of that reported in the above prior art, which is a significant advantage considering that at 100 mg and 200 mg dose strength modafinil is a high dose and consequently a large volume product. Thus the present invention solves a long felt need in the art for a process which overcomes these difficulties.

According to a first aspect of the present invention there is provided a novel process for preparing polymorphic form 5 of R-(−)-modafinil or S-(+)-modafinil, comprising the steps of:

(a) dissolving R-(−)-modafinil or S-(+)-modafinil in a solvent system; and
(b) recovering the modafinil.

Therefore, the present invention provides polymorphic form 5 of R-(−)-modafinil and S-(+)-modafinil as described according to the prior art above, that have higher optical and chemical purity.

For the purposes of the present invention, polymorphic form 5 of R-(−)-modafinil or S-(+)-modafinil is the same as form V disclosed in Cephalon's WO 2004/014846.

For the purposes of the present invention, the term “solvent system” means one solvent or a mixture of two or more solvents.

Preferably the solvent system used for the process for preparing polymorphic form 5 of R-(−)-modafinil or S-(+)-modafinil comprises:
(a) RaOCH2CH2ORb, wherein Ra and Rb are independently hydrogen or C1-4 alkyl, preferably wherein Ra and Rb are independently hydrogen, methyl or ethyl, more preferably wherein RaOCH2CH2ORb is ethylene glycol, 2-methoxy-ethanol, 2-ethoxy-ethanol or 1,2-dimethoxy-ethane; or
(b) RcOH, wherein Rc is C3-8 alkyl, preferably wherein Rc is C3-6 alkyl, more preferably wherein RcOH is 1-propanol, isopropanol, 1-butanol, 2-methyl-1-propanol, t-butanol, 1-pentanol, cyclopentanol, 1-hexanol, cyclohexanol, 1-heptanol or 1-octanol; or
(c) N,N-dimethylformamide; or
(d) a C4-10 alkane, preferably a C4-8 alkane, more preferably pentane, cyclopentane, hexane, cyclohexane or heptane; or
(e) toluene; or
(f) RdCOORe, wherein Rd is C1-6 alkyl, preferably wherein Rd is C1-4 alkyl, more preferably wherein Rd is methyl, and wherein Re is C3-6 alkyl, preferably wherein Re is C3-4 alkyl, more preferably wherein Re is n-propyl, most preferably wherein RdCOORe is n-propyl acetate; or
(g) an open-chain ether RfORg, wherein Rf and Rg are independently C1-6 alkyl, preferably wherein Rf and Rg are independently C1-4 alkyl, more preferably wherein Rf is methyl or ethyl and Rg is ethyl, propyl or butyl, most preferably wherein RfORg is diethyl ether or methyl t-butyl ether; or
(h) RhCORi, wherein Rh and Ri are each independently C1-8 alkyl, preferably C1-6 alkyl, and wherein RhCORi contains 4-12 carbon atoms, preferably 6-10 carbon atoms, preferably wherein RhCORi is ethyl methyl ketone or 4-methyl-pentan-2-one; or
(i) a mixture of two or more solvents selected from any of groups (a) to (h); water; methanol; ethanol; acetone; RjCOORk, wherein Rj is C1-6 alkyl, preferably wherein Rj is C1-4 alkyl, more preferably wherein Rj is methyl, and wherein Rk is methyl or ethyl, most preferably wherein RjCOORk is ethyl acetate; or a C3-8 cyclic ether such as tetrahydrofuran.

Preferably the solvent system comprises at least two solvents selected from: group (a) as defined above and C1-8 alcohols. Preferably the solvent system comprises at least two solvents selected from: ethylene glycol, 2-methoxy-ethanol, 2-ethoxy-ethanol, 1,2-dimethoxy-ethane, methanol, ethanol, 1-propanol, isopropanol, 1-butanol, 2-methyl-1-propanol, t-butanol, 1-pentanol, cyclopentanol, 1-hexanol, cyclohexanol, 1-heptanol and 1-octanol.

In a particularly preferred embodiment, the solvent system is selected from the solvent systems listed in Table 1 and Table 2.

According to a second aspect of the present invention there is provided a novel process for preparing polymorphic form 2 of R-(−)-modafinil or S-(+)-modafinil, comprising the steps of:

(a) dissolving R-(−)-modafinil or S-(+)-modafinil in a solvent system; and
(b) recovering the modafinil.

Therefore, the present invention provides polymorphic form 2 of R-(−)-modafinil and S-(+)-modafinil as described according to the prior art above, that have higher optical and chemical purity.

For the purposes of the present invention, polymorphic form 2 of R-(−)-modafinil or S-(+)-modafinil is the same as form II disclosed in Cephalon's U.S. Patent Publication No. 2006/0135621.

For the purposes of the present invention, the term “solvent system” means one solvent or a mixture of two or more solvents.

Preferably the solvent system used for the process for preparing polymorphic form 2 of R-(−)-modafinil or S-(+)-modafinil comprises:
(a) 2-methyl-1-propanol; or
(b) RdCOORe, wherein Rd is C1-6 alkyl, preferably wherein Rd is C1-4 alkyl, more preferably wherein Rd is methyl, and wherein Re is C3-6 alkyl, preferably wherein Re is C3-4 alkyl, more preferably wherein Re is n-propyl, most preferably wherein RdCOORe is n-propyl acetate; or
(c) a mixture of RlOH and RmCOORn, wherein Rl is C1-12 alkyl, preferably wherein Rl is C1-8 alkyl, preferably wherein Rl is C1-5 alkyl, more preferably wherein RlOH is methanol, ethanol, 1-propanol, isopropanol, 1-butanol, 2-methyl-1-propanol, t-butanol or 1-pentanol, most preferably wherein RlOH is ethanol, isopropanol or 2-methyl-1-propanol, and wherein Rm and Rn are independently C1-6 alkyl, preferably wherein Rm and Rn are independently C1-4 alkyl, more preferably wherein Rm and Rn are independently methyl, ethyl or propyl, most preferably wherein RmCOORn is ethyl acetate or n-propyl acetate.

Preferably the solvent system is selected from the group consisting of isopropanol (IPA), 2-methyl-1-propanol, n-propyl acetate, ethanol, ethyl acetate, and mixtures thereof.

In preferred embodiments of the first and second aspects of the present invention, the R-(−)-modafinil or S-(+)-modafinil is dissolved at the reflux temperature of the particular solvent system employed.

Preferably the R-(−)-modafinil or S-(+)-modafinil is dissolved in a small volume of the solvent system employed, preferably in a concentration of at least about 1 g/30 ml (30 vol), preferably at least about 1 g/20 ml (20 vol), preferably at least about 1 g/10 ml (10 vol), preferably at least about 1 g/5 ml (5 vol).

In preferred embodiments of the first and second aspects of the present invention, the modafinil is recovered as a precipitate. In a further embodiment, the precipitate is obtained by gradual cooling of the solution obtained in step (a). The gradual cooling of the modafinil-containing solution may result in the crystallization of particularly pure crystalline R-(−)-modafinil or S-(+)-modafinil. Accordingly, in a particularly preferred embodiment according to any of the above aspects of the processes, the cooling rate ranges from about 0.3 deg/min to about 1.8 deg/min. Particularly preferred is a range from about 1 deg/min to about 1.5 deg/min. For the purposes of the present invention, when referring to “deg”, what is meant is degree centigrade. Alternatively, the precipitate is obtained by the addition of an anti-solvent to the solution obtained in step (a).

In preferred embodiments of the first and second aspects of the present invention, the modafinil is obtained on an industrial scale, preferably in batches of 0.5 kg, 1 kg, 5 kg, 10 kg, 50 kg, 100 kg, 500 kg, 1000 kg or more.

In one aspect according to the invention there is provided a polymorphic form 5 of R-(−)-modafinil or S-(+)-modafinil that is substantially free of other polymorphs.

A further aspect comprises a polymorphic form 2 of R-(−)-modafinil or S-(+)-modafinil that is substantially free of other polymorphs.

For the purposes of the present invention, “substantially free of other polymorphs” means that the polymorphic form in question comprises less than 90% of other polymorphic forms, preferably less than 95%, preferably less than 96%, preferably less than 97%, preferably less than 98%, and more preferably less than 99%.

A yet further aspect provides a polymorphic form 5 of R-(−)-modafinil or S-(+)-modafinil with greater than or equal to 90% optical and 90% chemical purity. A still further aspect provides a polymorphic form 5 wherein the optical and/or chemical purity is greater than or equal to 95%, preferably greater than or equal to 96%, preferably greater than or equal to 97%, preferably greater than or equal to 98%, and preferably greater than or equal to 99%.

Another aspect relates to a polymorphic form 2 of R-(−)-modafinil or S-(+)-modafinil with greater than or equal to 90% optical and 90% chemical purity. Yet another aspect provides a polymorphic form 2 wherein the optical and/or chemical purity is greater than or equal to 95%, preferably greater than or equal to 96%, preferably greater than or equal to 97%, preferably greater than or equal to 98%, and preferably greater than or equal to 99%.

For the purposes of the present invention, when referring to R-(−)-modafinil or S-(+)-modafinil, what is meant is substantially enantiomerically or optically or chirally pure modafinil. The terms “enantiomeric”, “optical” or “chiral” are used interchangeably herein. “Substantially enantiomerically pure”, “substantially optically pure” or “substantially chirally pure” means that the modafinil comprises at least 90%, preferably 91%, preferably 92%, preferably 93%, preferably 94%, preferably 95%, preferably 96%, preferably 97%, preferably 98%, and preferably 99% of the desired enantiomer.

For the purposes of the present invention, polymorphic purity is preferably measured by XRPD or DSC. Enantiomeric or optical or chiral purity is preferably measured by chiral HPLC. Chemical purity is preferably measured by HPLC.

In a further aspect according to the invention, there is provided a pharmaceutical composition comprising modafinil form 2 or form 5 according to the present invention or prepared according to a process of the present invention.

In yet another aspect there is provided a use of a pharmaceutical composition according to the present invention for the treatment or prevention of one or more of narcolepsy, obstructive sleep apnoea/hypopnoea syndrome (OSAHS), and moderate to severe chronic shift work sleep disorder (SWSD).

### DETAILED DESCRIPTION OF THE INVENTION

The structure of modafinil includes a stereocentre and, therefore, modafinil can exist as a racemate, one of two pure enantiomers, or a mixture of the two enantiomers in any ratio.

In the pharmaceutical industry, polymorphic control of an active pharmaceutical ingredient (API) is critical, since polymorphs have different chemical and physical stability, solubility, morphology, and hygroscopicity. During the manufacturing process, it is often required to convert a less stable form to a more stable form.

As used herein and unless otherwise specified, the terms “enantiomerically pure” or “chiral purity” include a composition which is substantially enantiomerically pure and include, for example, a composition with greater than or equal to about 90%, 91%, 92%, 93%, 94%, 95%, 96%, 97%, 98%, or 99% presence of the relevant enantiomeric form.

The crystalline forms of a given compound generally have physical, pharmaceutical, physiological and biological properties, which differ from each other very sharply. In this respect the crystalline forms of optically active modafinil are of interest in that they have different and advantageous properties.

Enantiomers are typically designated using either (+) and (−), or (d) and (l), which indicates optical rotating power in the chiral centre. Stereoisomerism may also be denoted by either (D) or (L), or by (R) and (S), these being descriptive of the absolute configuration. In what follows, the levorotatory enantiomer of modafinil will be referred to as the R-(−)-, R- or (−)-enantiomer, and the dextrorotatory enantiomer will for its part be referred to as the S-(+)-, S- or (+)-enantiomer.

The terms “crystalline form” and “polymorph” or “polymorphic form” are used interchangeably herein.

As used herein, the term “modafinil” includes the racemate, other mixtures of the R- and S-isomers, and single enantiomers, but may be specifically set forth as the racemate, R-isomer, S-isomer, or any mixture of both R- and S-isomers.

The present invention relates to a process for the preparation of polymorphic form 2 and polymorphic form 5 of R-(−)-modafinil, and polymorphic form 2 and polymorphic form 5 of S-(+)-modafinil, which are substantially free of other known polymorphs and, in particular, of the corresponding enantiomer. The process provides R- and S-modafinil with high optical and chemical purity. The process for making these polymorphs is simple and reproducible. Further the process of the present invention is amenable to scale up and the polymorph has a uniform crystallinity. The inventors have found that the gradual cooling of the solvent comprising the modafinil of any polymorphic form results in the desired form having an excellent chiral and chemical purity profile. It has been found that a cooling rate of about 0.3 deg/min to about 2 deg/min, particularly about 0.5 deg/min to about 1.5 deg/min is particularly advantageous. For the purposes of the present invention, when referring to “deg”, what is meant is degree centigrade. However it will be apparent to the skilled person that the said cooling rate is not to be limited, but may be varied and remain within the scope and spirit of the invention.

It will of course be understood by the skilled person that the R-(−)-modafinil or S-(+)-modafinil prepared according to the processes of the invention may be incorporated in pharmaceutical compositions. Such compositions may be solid, such as tablets, pellets, or capsules, or liquid compositions, and may further comprise pharmaceutically acceptable excipients suitable for the required dosage form.

Certain embodiments of solid pharmaceutical compositions according to the invention may include immediate release compositions, or controlled release compositions including modified and sustained release. Preferred embodiments further comprise suitable excipients that aid in the manufacture and stability of the composition. In a preferred embodiment, compositions according to the invention further comprise a diluent, glidant, antioxidant, buffering agent, coating agent, flavourant, lubricant, binder and/or filler. These excipients can be any type typically used in the art of pharmaceutical formulations.

In further embodiments still the modafinil may be particulate in nature, being either coated or uncoated.

The following paragraphs enumerated consecutively from 1 through 28 provide for various aspects of the present invention. In one embodiment, the present invention provides:

1. A process for preparing polymorphic form 5 of R-(−)-modafinil or S-(+)-modafinil, comprising the steps of:
(a) dissolving R-(−)-modafinil or S-(+)-modafinil in a solvent system; and
(b) recovering the modafinil.
   2. The process according to paragraph 1, wherein the solvent system comprises:
(a) RaOCH2CH2ORb, wherein Ra and Rb are independently hydrogen or C1-4 alkyl; or
(b) RcOH, wherein Rc is C3-8 alkyl; or
(c) N,N-dimethylformamide; or
(d) a C4-10 alkane; or
(e) toluene; or
(f) RdCOORe, wherein Rd is C1-6 alkyl, and wherein Re is C3-6 alkyl; or
(g) an open-chain ether RfORg, wherein Rf and Rg are independently C1-6 alkyl; or
(h) RhCORi, wherein Rh and Rl are each independently C1-8 alkyl, and wherein RhCORi contains 4-12 carbon atoms; or
(i) a mixture of two or more solvents selected from any of groups (a) to (h); water; methanol; ethanol; acetone; RjCOORk wherein Rj is C1-6 alkyl and wherein Rk is methyl or ethyl; or a C3-8 cyclic ether.
   3. The process according to paragraph 2, wherein the solvent system comprises at least two solvents selected from: group (a) and C1-8 alcohols.
   4. The process according to any one of paragraphs 1 to 3, wherein the solvent system comprises at least two solvents selected from: ethylene glycol, 2-methoxy-ethanol, 2-ethoxy-ethanol, 1,2-dimethoxy-ethane, methanol, ethanol, 1-propanol, isopropanol, 1-butanol, 2-methyl-1-propanol, t-butanol, 1-pentanol, cyclopentanol, 1-hexanol, cyclohexanol, 1-heptanol and 1-octanol.
   5. The process according to any one of paragraphs 1 to 4, wherein the solvent system is selected from the solvent systems listed in Table 1 and Table 2.
   6. A process for preparing polymorphic form 2 of R-(−)-modafinil or S-(+)-modafinil, comprising the steps of:
(a) dissolving R-(−)-modafinil or S-(+)-modafinil in a solvent system; and
(b) recovering the modafinil.
   7. The process according to paragraph 6, wherein the solvent system comprises:
(a) 2-methyl-1-propanol; or
(b) RdCOORe, wherein Rd is C1-6 alkyl, and wherein Re is C3-6 alkyl; or
(c) a mixture of RlOH and RmCOORn, wherein Rl is C1-12 alkyl, and wherein Rm and Rn are independently C1-6 alkyl.
   8. The process according to paragraph 6 or 7, wherein the solvent system is selected from: isopropanol (IPA), 2-methyl-1-propanol, n-propyl acetate, ethanol, ethyl acetate, or a mixture thereof.
   9. The process according to any one of the preceding paragraphs, wherein the R-(−)-modafinil or S-(+)-modafinil is dissolved at the reflux temperature of the particular solvent system employed.
   10. The process according to any one of the preceding paragraphs, wherein the modafinil is recovered as a precipitate.
   11. The process according to paragraph 10, wherein the precipitate is formed by cooling the solution.
   12. The process according to paragraph 10, wherein the precipitate is formed by adding an anti-solvent to the solution obtained in step (a).
   13. The process according to any one of paragraphs 10 to 12, wherein the modafinil precipitate is recovered by filtration.
   14. The process according to paragraph 11, wherein the cooling rate ranges from about 0.3 deg/min to about 1.8 deg/min.
   15. The process according to paragraph 14, wherein the range is from about 1 deg/min to about 1.5 deg/min.
   16. Polymorphic form 5 or polymorphic form 2 of R-(−)-modafinil or S-(+)-modafinil prepared by a process according to any one of the preceding paragraphs.
   17. Polymorphic form 5 of R-(−)-modafinil or S-(+)-modafinil that is substantially free of other polymorphs.
   18. Polymorphic form 2 of R-(−)-modafinil or S-(+)-modafinil that is substantially free of other polymorphs.
   19. Polymorphic form 5 of R-(−)-modafinil or S-(+)-modafinil with greater than or equal to 90% optical and/or 90% chemical purity.
   20. Polymorphic form 5 according to paragraph 19, wherein the optical and/or chemical purity is greater than or equal to 99%.
   21. Polymorphic form 2 of R-(−)-modafinil or S-(+)-modafinil with greater than or equal to 90% optical and/or 90% chemical purity.
   22. Polymorphic form 2 according to paragraph 21, wherein the optical and/or chemical purity is greater than or equal to 99%.
   23. Modafinil according to any one of paragraphs 16 to 22, for use in medicine.
   24. Modafinil according to any one of paragraphs 16 to 23, for treating or preventing narcolepsy, obstructive sleep apnoea/hypopnoea syndrome (OSAHS), or shift work sleep disorder (SWSD).
   25. A pharmaceutical composition comprising modafinil according to any one of paragraphs 16 to 24, and further comprising one or more pharmaceutically acceptable excipients.
   26. The pharmaceutical composition according to paragraph 25, for treating or preventing narcolepsy, obstructive sleep apnoea/hypopnoea syndrome (OSAHS), or shift work sleep disorder (SWSD).
   27. Use of modafinil according to any one of paragraphs 16 to 24 in the manufacture of a medicament for the treatment or prevention of narcolepsy, obstructive sleep apnoea/hypopnoea syndrome (OSAHS), or shift work sleep disorder (SWSD).
   28. A method of treating or preventing narcolepsy, obstructive sleep apnoea/hypopnoea syndrome (OSAHS), or shift work sleep disorder (SWSD), comprising administering to a person in need thereof a pharmaceutical composition comprising a therapeutically or prophylactically effective amount of modafinil according to any one of paragraphs 16 to 24.

The details of the invention, its objects and advantages are explained hereunder in greater detail in the following non-limiting examples.

### EXAMPLES

### Example 1

Preparation of enantiomerically and chemically pure R-(−)-modafinil form 5 2 g of R-(−)-modafinil was heated to 55° C. in 1-pentanol (10 ml, 5 vol) and isopropanol (10 ml, 5 vol) to obtain a clear solution. The clear solution was filtered hot through a Buchner funnel. The filtrate was cooled slowly to 25° C. within 50-60 minutes. A solid precipitated out at this temperature. Stirring was maintained at this temperature for 1.5 hours. The solid was filtered and dried under vacuum at 40° C. for 2 hours.

Yield=74%

Chiral purity=99.9% (as measured by chiral HPLC)

Chemical purity=99.69% (as measured by HPLC)

Polymorphic purity>99% (as measured by DSC)

Table 1 summarises examples 2-15 prepared according to the process described in example 1 with the starting materials and reaction conditions as shown. In all of examples 2-15, the R-(−)-modafinil was heated in the solvent system to about 80° C. to obtain a clear solution (heating temperature). In all of examples 2-15, the precipitated solid was filtered at about 25-30° C. (filtration temperature).

TABLE 1

| Ex- | | | | | |
|---|---|---|---|---|---|
| am- | Modafinil | Modafinil | | Vol | Form |
| ple | input (g) | output (g) | Solvents | (ml) | obtained |
| 2 | 2.0 | 0.86 | ethylene glycol/ | 5/5 | Form 5 |
| | | | methanol | | |
| 3 | 2.0 | 0.84 | ethylene glycol/ | 2.5/5 | Form 5 |
| | | | isopropanol | | |
| 4 | 2.0 | 0.80 | ethylene glycol/ | 2.5/2.5 | Form 5 |
| | | | ethanol | | |
| 5 | 2.0 | 0.75 | ethylene glycol/ | 2.5/2.5 | Form 5 |
| | | | 1-pentanol | | |
| 6 | 2.0 | 1.20 | ethylene glycol/ | 2.5/2.5 | Form 5 |
| | | | cyclohexanol | | |
| 7 | 2.0 | 0.70 | ethylene glycol/ | 2.5/2.5 | Form 5 |
| | | | 1-butanol | | |
| 8 | 2.0 | 1.40 | 2-methoxy-ethanol/ | 2.5/2.5 | Form 5 |
| | | | isopropanol | | |
| 9 | 2.0 | 1.00 | 2-methoxy-ethanol/ | 2.5/2.5 | Form 5 |
| | | | ethanol | | |
| 10 | 2.0 | 1.20 | 2-methoxy-ethanol/ | 2.5/2.5 | Form 5 |
| | | | methanol | | |
| 11 | 2.0 | 0.89 | 2-methoxy-ethanol/ | 2.5/2.5 | Form 5 |
| | | | cyclohexanol | | |
| 12 | 2.0 | 1.69 | 2-methoxy-ethanol/ | 2.5/2.5 | Form 5 |
| | | | 1-butanol | | |
| 13 | 2.0 | 1.60 | 2-methoxy-ethanol/ | 2.5/2.5 | Form 5 |
| | | | 1-pentanol | | |
| 14 | 2.0 | 1.59 | 2-methoxy-ethanol/ | 2.5/2.5 | Form 5 |
| | | | 2-ethoxy-ethanol | | |
| 15 | 2.0 | 1.56 | 2-methoxy-ethanol/ | 2.5/2.5 | Form 5 |
| | | | 2-methyl-1- | | |
| | | | propanol | | |

Table 2 below shows a non-exhaustive list of further solvent systems that could be employed in the preparation of form 5 of R-(−)- or S-(+)-modafinil.

TABLE 2

| | |
|---|---|
| 2-ethoxy-ethanol/2-methyl-1-propanol | 2-methyl-1-propanol/1-pentanol |
| methanol/water | dimethylformamide |
| | (DMF)/water |
| tetrahydrofuran (THF)/water | t-butanol/n-hexane/water |
| acetone/water | ethanol/toluene/water |
| ethyl methyl ketone/n-hexane | ethanol/heptane/water |
| 1-pentanol/water | 2-methyl-1-propanol/methanol |
| ethyl methyl ketone/1-pentanol | 1-pentanol/ethanol |
| ethyl methyl ketone/2-methoxy-ethanol | ethanol/1-butanol |
| ethyl methyl ketone/n-propyl acetate | ethanol/n-propyl acetate |
| ethyl methyl ketone/1-butanol | ethanol/1,2-dimethoxy-ethane |
| 1-pentanol/methanol | 2-ethoxy-ethanol/water |
| 1-butanol/methanol | 1,2-dimethoxy-ethane/water |
| 2-ethoxy-ethanol/methanol | cyclopentanol/water |
| 4-methyl-pentan-2-one/ethanol | ethylene glycol/water |
| isopropanol/2-methyl-1-propanol | octanol/water |
| isopropanol/n-propyl acetate | methanol/n-hexane |
| isopropanol/4-methyl-pentan-2-one | water/n-hexane |
| isopropanol/1-pentanol | methanol/methyl t-butyl ether |
| | (TBME) |
| isopropanol/1-butanol | 2-methyl-1-propanol/t-butanol |
| 1-pentanol/methyl t-butyl ether (TBME) | 2-methyl-1-propanol/isopropanol |
| 1-butanol/methyl t-butyl ether (TBME) | isopropanol/toluene |
| octanol/diethyl ether | ethanol/cyclohexane |
| 2-methyl-1-propanol/1-butanol | ethanol/n-heptane |

### Example 16

Preparation of enantiomerically and chemically pure R-(−)-modafinil form 2 2 g of R-(−)-modafinil was heated to 80° C. in n-propyl acetate (60 ml, 30 vol) to obtain a clear solution. The clear solution was filtered hot through a Buchner funnel. The filtrate was cooled slowly to 25° C. within 2 hours. A solid precipitated out at this temperature. Stirring was maintained at this temperature for 1.5 hours. The solid was filtered and dried under vacuum at 40° C. for 2 hours.

Yield=34%

Chiral purity=99.83% (as measured by chiral HPLC)

Chemical purity=99.88% (as measured by chiral HPLC)

Polymorphic purity>99% (as measured by DSC)

Table 3 summarises examples 17 and 18 prepared according to the process described in example 16 with the starting materials and reaction conditions as shown.

TABLE 3

| | | | | | Heat- | Fil- | |
|---|---|---|---|---|---|---|---|
| | Modafinil | | | | ing | tration | Form |
| | input | Modafinil | | Vol | temp. | temp.* | ob- |
| Ex. | (g) | output (g) | Solvents | (ml) | (° C.) | (° C.) | tained |
| 17 | 2.0 | 1.72 | n-propyl | 5 | 80 | 25 | 2 |
| | | | acetate | | | | |
| 18 | 2.0 | 0.32 | ethanol/ | 5/5 | 64 | 5 | 2 |
| | | | ethyl | | | | |
| | | | acetate | | | | |

*Filtration temperature refers to the temperature at which the precipitated solid was filtered.

1. A process for preparing polymorphic form 5 of R-(−)-modafinil or S-(+)-modafinil, comprising the steps of:
(a) dissolving R-(−)-modafinil or S-(+)-modafinil in a solvent system; and
(b) recovering the modafinil.

2. The process according to claim 1, wherein the solvent system comprises:
(a) R^{a}OCH₂CH₂OR^{b}, wherein R^{a} and R^{b} are independently hydrogen or C₁₋₄ alkyl; or
(b) R^{c}OH, wherein R^{c} is C₃₋₈ alkyl; or
(c) N,N-dimethylformamide; or
(d) a C₄₋₁₀ alkane; or
(e) toluene; or
(f) R^{d}COOR^{e}, wherein R^{d} is C₁₋₆ alkyl, and wherein R^{e} is C₃₋₆ alkyl; or
(g) an open-chain ether R^{f}OR^{g}, wherein R^{f} and R^{g} are independently C₁₋₆ alkyl; or
(h) R^{h}CORⁱ, wherein R^{h} and Rⁱ are each independently C₁₋₈ alkyl, and wherein R^{h}CORⁱ contains 4-12 carbon atoms; or
(i) a mixture of two or more solvents selected from any of groups (a) to (h); water; methanol; ethanol; acetone; R^{j}COOR^{k} wherein R^{j} is C₁₋₆ alkyl and wherein R^{k} is methyl or ethyl; or a C₃₋₈ cyclic ether.

3. The process according to claim 2, wherein the solvent system comprises at least two solvents selected from: group (a) and C₁₋₈ alcohols.

4. The process according to any one of claims 1 to 3, wherein the solvent system comprises at least two solvents selected from: ethylene glycol, 2-methoxy-ethanol, 2-ethoxy-ethanol, 1,2-dimethoxy-ethane, methanol, ethanol, 1-propanol, isopropanol, 1-butanol, 2-methyl-1-propanol, t-butanol, 1-pentanol, cyclopentanol, 1-hexanol, cyclohexanol, 1-heptanol and 1-octanol.

5. The process according to any one of claims 1 to 4, wherein the solvent system is selected from the solvent systems listed in Table 1 and Table 2.

6. A process for preparing polymorphic form 2 of R-(−)-modafinil or S-(+)-modafinil, comprising the steps of:
(a) dissolving R-(−)-modafinil or S-(+)-modafinil in a solvent system; and
(b) recovering the modafinil.

7. The process according to claim 6, wherein the solvent system comprises:
(a) 2-methyl-1-propanol; or
(b) R^{d}COOR^{e}, wherein R^{d} is C₁₋₆ alkyl, and wherein R^{e} is C₃₋₆ alkyl; or
(c) a mixture of R^{l}OH and R^{m}COORⁿ, wherein R^{l} is C₁₋₁₂ alkyl, and wherein R^{m} and Rⁿ are independently C₁₋₆ alkyl.

8. The process according to claim 6 or 7, wherein the solvent system is selected from: isopropanol (IPA), 2-methyl-1-propanol, n-propyl acetate, ethanol, ethyl acetate, or a mixture thereof.

9. The process according to any one of the preceding claims, wherein the R-(−)-modafinil or S-(+)-modafinil is dissolved at the reflux temperature of the particular solvent system employed.

10. The process according to any one of the preceding claims, wherein the modafinil is recovered as a precipitate.

11. The process according to claim 10, wherein the precipitate is formed by cooling the solution.

12. The process according to claim 10, wherein the precipitate is formed by adding an anti-solvent to the solution obtained in step (a).

13. The process according to any one of claims 10 to 12, wherein the modafinil precipitate is recovered by filtration.

14. The process according to claim 11, wherein the cooling rate ranges from about 0.3 deg/min to about 1.8 deg/min.

15. The process according to claim 14, wherein the range is from about 1 deg/min to about 1.5 deg/min.

16. Polymorphic form 5 or polymorphic form 2 of R-(−)-modafinil or S-(+)-modafinil prepared by a process according to any one of the preceding claims.

17. Polymorphic form 5 of R-(−)-modafinil or S-(+)-modafinil that is substantially free of other polymorphs.

18. Polymorphic form 2 of R-(−)-modafinil or S-(+)-modafinil that is substantially free of other polymorphs.

19. Polymorphic form 5 of R-(−)-modafinil or S-(+)-modafinil with greater than or equal to 90% optical and/or 90% chemical purity.

20. Polymorphic form 5 according to claim 19, wherein the optical and/or chemical purity is greater than or equal to 99%.

21. Polymorphic form 2 of R-(−)-modafinil or S-(+)-modafinil with greater than or equal to 90% optical and/or 90% chemical purity.

22. Polymorphic form 2 according to claim 21, wherein the optical and/or chemical purity is greater than or equal to 99%.

23. Modafinil according to any one of claims 16 to 22, for use in medicine.

24. Modafinil according to any one of claims 16 to 23, for treating or preventing narcolepsy, obstructive sleep apnoea/hypopnoea syndrome (OSAHS), or shift work sleep disorder (SWSD).

25. A pharmaceutical composition comprising modafinil according to any one of claims 16 to 24, and further comprising one or more pharmaceutically acceptable excipients.

26. The pharmaceutical composition according to claim 25, for treating or preventing narcolepsy, obstructive sleep apnoea/hypopnoea syndrome (OSAHS), or shift work sleep disorder (SWSD).

27. Use of modafinil according to any one of claims 16 to 24 in the manufacture of a medicament for the treatment or prevention of narcolepsy, obstructive sleep apnoea/hypopnoea syndrome (OSAHS), or shift work sleep disorder (SWSD).

28. A method of treating or preventing narcolepsy, obstructive sleep apnoea/hypopnoea syndrome (OSAHS), or shift work sleep disorder (SWSD), comprising administering to a person in need thereof a pharmaceutical composition comprising a therapeutically or prophylactically effective amount of modafinil according to any one of claims 16 to 24.
